(19) United States
(12) Patent Application Publication
(10) Pub. No.: US 2010/0286712 A1
(43) Pub. Date: Nov. 11, 2010

(54) BED INTEGRATED SURGICAL ROBOT

(76) Inventors: Jong Seok Won, Gyeonggi-do (KR); Seung Wook Choi, Gyeonggi-do (KR); William Peine, Ashland, MA (US)

Correspondence Address: BIRCH STEWART KOLASCH & BIRCH, PO BOX 747, FALLS CHURCH, VA, 22040-0747, US

(21) Appl. No.: 12/812,425

(22) PCT Filed: Oct. 7, 2008
(86) PCT No.: PCT/KR08/05864

§ 371 Date: Jul. 9, 2010

(30) Foreign Application Priority Data
Feb. 20, 2008 (KR) 10-2008-0015087

(51) Int. Cl. A61B 19/00

(52) U.S. Cl. 606/130

(57) ABSTRACT

Disclosed is a bed integrated surgical robot. The bed integrated surgical robot comprises a supporting part, a frame supported by the supporting part, a table supported by the frame and on which a surgical patient is laid and a surgical robot arm installed on the frame and manipulated to reach for the surgical patient, wherein the robot arm is formed of a plurality of joints and deposited on the frame by driving the plurality of joints, and is easy to deposit, install, move, allows an assistant surgeon to easily access a patient under surgery, requires no additional space to install the robot, and functions as a patient bed.

### CROSS-REFERENCE TO RELATED APPLICATIONS

This application claims foreign priority benefits under 35 U.S.C. sctn. 119(a)-(d) to PCT/KR2008/005864, filed Oct. 7, 2008, which is hereby incorporated by reference in its entirety.

### BACKGROUND

The present invention relates to a surgical robot integrated with patient bed.

Surgery refers to a medical specialty that uses operative manual and instrumental techniques on the tissues of a patient to treat a pathological condition. Surgical robots have been proposed as an alternative for performing an excision surgery, which needs cutting tissues to treat or remove the organ within the body, to reduce blood loss, pain and improve precision.

The surgical robot consists of a master robot generating and transmitting signals according to manipulations of a surgeon and a slave robot applying the manipulation directly to the patient according to the signals from the master robot. The master robot may be integrated with the slave robot or may be separated from the slave robot.

However, despite of the rapid advance in the surgical robot, the surgical robot is not in active use due to high price and maintenance costs, large volume, lack of skilled surgeons, and reliability. Especially, the robot arm which should be extended to a surgery table is a main cause of the large volume.

A surgical robot having a large volume is space consuming and inconvenient for moving, thereby unsuitable for small hospitals. Also, the large volume robot makes it difficult for an assistant surgeon to access the patient, which in an emergency may threaten the life of the patient.

### SUMMARY

The present invention aims to provide a small and lightweight bed integrated surgical robot that is easy to deposit, install and move, space-saving, and functions as a surgical bed as well.

According to one aspect of the present invention is provided a bed integrated surgical robot comprising a supporting part, a frame supported by the supporting part, a table supported by the frame and on which a surgical patient is laid and a surgical robot arm installed on the frame and manipulated to reach for the surgical patient, wherein the robot arm is formed of a plurality of joints and deposited on the frame by driving the plurality of joints.

According to another aspect of the present invention is provided a bed integrated surgical robot comprising a supporting part, a frame supported by the supporting part, a table supported by the frame and on which a surgical patient is laid and a surgical robot arm installed on the frame and manipulated to reach for the surgical patient, wherein the robot arm is formed of a plurality of joints and a fore end of the robot arm reaches for a predetermined area of the table by driving the plurality of joints.

The supporting part may comprise a moving means for moving the robot.

The frame may be combined with a height adjustment means for adjusting a height of the table.

A height of the robot arm may be adjusted in synchronization with the table.

The table may be formed of a plurality of sub tables and a pivot means may be combined with the frame to separately pivot the plurality of sub tables in correspondence with a posture of the patient.

The robot arm may be pivoted in synchronization with the sub table, whereby the fore end of the robot arm reaches for a predetermined area of the sub table.

The surgical robot may further comprise a main body part that is connected with the robot arm and transmits signal for driving the plurality of joints.

The main body part may transmit signal corresponding to a deposit mode, and when the robot arm receives the signal corresponding to the deposit mode the plurality of joints are driven to deposit the robot arm on the frame.

The main body part may transmit signal corresponding to an operation mode, and when the robot arm receives the signal corresponding to the operation mode the plurality of joints are driven such that the fore end of the robot arm reaches for a predetermined area of the table.

Additional aspects, features, and advantages will be elucidated from the following drawings, claims, and specification.

The present invention provides a compact size surgical robot that is easy to deposit, install and move. The surgical robot according to this invention allows an assistant surgeon to easily access a patient under surgery, requires no additional space to install the robot, and functions as a patient bed.

### BRIEF DESCRIPTION OF THE DRAWINGS

FIG. 1 is a perspective view of a surgical robot integrated with patient bed according to an embodiment of the present invention.

FIGS. 2 through 5 are schematic views showing a surgical robot arm is deposited on a frame in accordance with the embodiment of the present invention.

FIG. 6 is a perspective view of a surgical robot integrated with patient bed according to another embodiment of the present invention.

### DETAILED DESCRIPTION

Although a few embodiments of the present invention have been shown and described, it will be appreciated by those skilled in the art that changes may be made in these embodiments without departing from the principles and spirit of the invention, the scope of which is defined in the appended claims and their equivalents. Also, specific descriptions on related prior art will be omitted in order to concentrate on the gist of the present invention.

The terms first, second, third and the like in the description and in the claims, are used for distinguishing between similar elements and not necessarily for describing a sequential or chronological order.

The present invention will be described with respect to particular embodiments and with reference to certain drawings but the invention is not limited thereto only by the claims. Where an indefinite or definite article is used when referring to a singular noun e.g. “a” or “an”, “the”, this includes a plural of that noun unless something else is specifically stated.

It is to be noticed that the term “comprising”, used in the claims, should not be interpreted as being restricted to the means listed thereafter; it does not exclude other elements or steps. It is thus to be interpreted as specifying the presence of the stated features, integers, steps or components as referred to, but does not preclude the presence or addition of one or more other features, integers, steps or components, or groups thereof.

Hereinafter, same reference characters designate the same or similar parts throughout the drawings and the repeated description about the same reference characters is omitted.

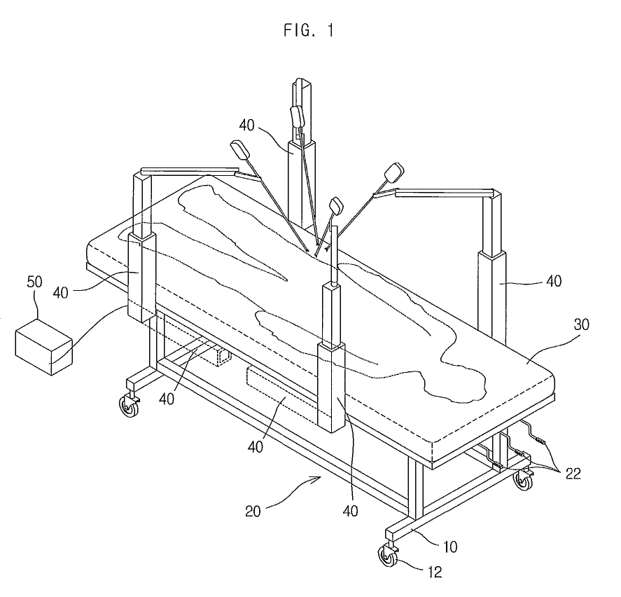

FIG. 1 is a perspective view of a surgical robot integrated with patient bed according to an embodiment of the present invention. In FIG. 1 are illustrated a supporting part 10, a moving means 12, a frame 20, a height adjustment means 22, a table 30, a robot arm 40, and a main body part 30.

This embodiment has a feature in that the robot arms 40 are integrated with an operating table, and the robot arms 40 can be deposited under the table 30 so that the surgeon can access, place. and remove the patient without separating the surgical robot from the operating table.

The bed integrated robot according to the present embodiment comprises the supporting part 10, the frame 20, and the table 30. The table 30 on which the patient is placed is supported by the frame 20, and the frame 20 is supported by the supporting part 10.

The bed integrated robot has a feature in that the robot arm 40 is installed on the frame 20, such that the robot arm 40 formed of several joints extends and/or pivots to perform an operation on the patient.

In order for the extension may be applied to the robot arm 40 an extension system of slide type, telescope type or the like, and in order for pivoting may be applied a pivoting system such as a pivoting axis, a link and the like.

While the robot is on operation, the robot arm 40 is pivot and extended from the operating table as shown in FIG. 1 so that a surgical instrument combined at an end of the robot arm 40 reaches the wound of the patient.

While the robot is off operation, the robot arm 40 is retracted and pivot to be deposited under the table 30, namely on the frame 20.

When the robot arm 40 is deposited under the operating table, the surgical robot can be installed without consuming extra space.

In this way, by reducing the volume of the surgical robot, surgical robots can be introduced in small hospitals and loaded in an ambulance and elevator, which helps manage emergency.

In order to retract the robot arm 40 may be employed a hinge, slide, rotation axis, or the like, the detail of which will be described later.

Also, the bed integrated robot has a feature in that a patient bed is functionally integrated with a surgical robot. Since the robot arm 40 is installed on the frame 20 of the bed, a coordinate origin of the robot arm 40 corresponds to a predetermined position of the frame 20.

Accordingly, the robot arm 40 may be manipulated such that the instrument reaches a object area of the table 30 or the patient without inputting information on position relation between the robot and the bed or between the robot and the patient.

Since a driving coordinate of the robot arm 40 is associated with the bed, the coordinate remains unchanged, so that a surgeon can perform an operation even when the bed is moving or trembling.

The supporting part 10 may have the moving means 12 such as wheels or rollers for moving the bed.

The frame 20 of the bed may have the height adjustment means 22 that can adjust the height of the table 30. Examples of the height adjustment means 22 include a handle, screw, a worm gear, and the like.

The height of the robot arm 40 may also be adjusted in synchronization with the height of the table 30, whereby the robot arm 40 can be manipulated on the basis of the same coordinate even though the height of the table 4 changes since the driving coordinate of the robot arm 40 is uninfluenced by the height adjustment.

In order to synchronize the robot arm 40 with the height of the table 30, the robot arm 40 may be connected with the table 30 such that the height of the robot arm 40 is adjusted in accordance with the height adjustment of the table 30, or an input value corresponding to the adjusted height of the table 30 may be stored so that the coordinate is changed according to the inputted value.

In the case that the surgical robot of the present embodiment is employed as a slave robot, a master robot may transmit signals for manipulating the robot arm 40, and when a separate interface is disposed between the master robot and the slave robot, the signals are transmitted to the slave from the interface.

In the below, the main body part 50 refers to a system that transmits signals to the robot arm 40, example of which is a master robot or an interface system.

The bed integrated surgical robot according to the present invention is deposited under the bed before set for an operation mode, so that the main body part 50 provides the slave robot with signals corresponding to the deposit mode and the operation mode.

Accordingly, the robot arm 40 is converted to the deposit or operation mode by driving the joints according to the signals.

In the case that a signal corresponding to the deposit mode is provided from the main body part 50, the robot arm 40 is shortened and pivoted to be deposited under the table 30, on the frame 20 on which the robot arm 40 is installed, as shown in dotted line in FIG. 1.

In the case that a signal corresponding to the operation mode is provided, the robot arm 40 is pivoted and extended such that the instrument at the fore end of the robot arm 40 reaches the patient.

As described above, by depositing and withdrawing the robot arm 40 under/from the bed, the drawback in the prior slave robot, difficulty to access patient during surgery and difficulty to disassemble under emergency, may be overcome.

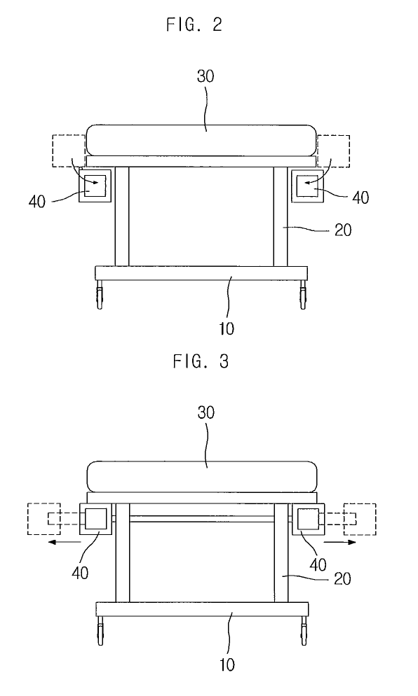

FIGS. 2 through 5 are schematic views showing a surgical robot arm is deposited on a frame in accordance with the embodiment of the present invention. In FIGS. 2 through 5 are illustrated a supporting part 10, a frame 20, a table 30 and a robot arm 40.

FIGS. 2 through 5 show sample methods by which the robot arm 40 is deposited.

FIG. 2 introduces a method in which the robot arm 40 is shortened to a predetermined length before deposited under the table 30. According to this method, a base of the robot arm 40 forms a hinge combination with an end of the frame so that the robot arm 40 pivots to be deposited on the frame 20 under the table 30. The depositing may be a part of the driving mechanism of the robot arm 40 or performed manually.

FIG. 3 shows another method in which a base of the robot arm 40 is deposited and withdrawn by a slide motion. Like in the method of FIG. 2, the depositing may be a part of the driving mechanism of the robot arm 40 or performed manually.

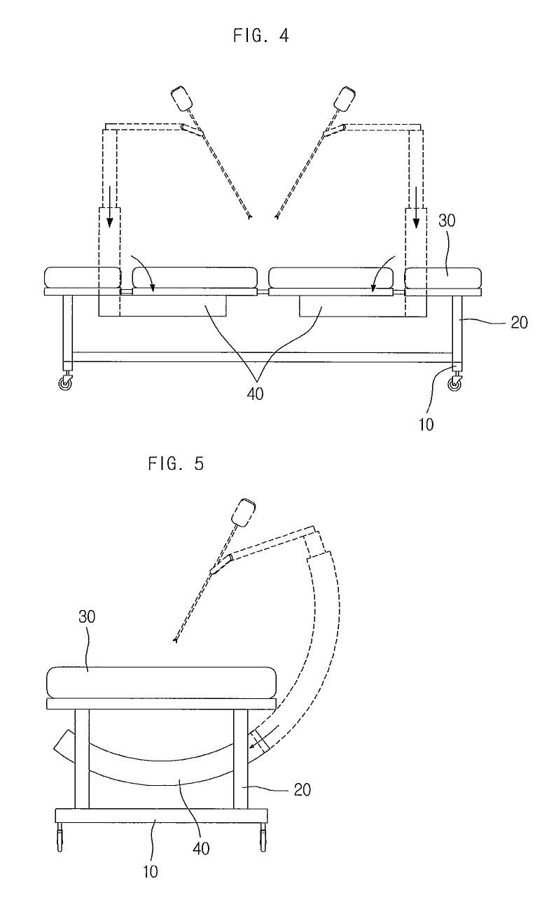

FIG. 4 shows a mechanism by which the robot arm 40 finished with a surgery pivots to a ‘deposit capable state’ that is illustrated in dotted line in FIGS. 2 and 3. The fore end of the robot arm 40 is accommodated in the base through a telescope method or the like, through which the length of the robot arm 40 is shortened, and the base rotates about an axis installed in the frame 20, forming the ‘deposit capable state’.

FIG. 5 shows another method in which a base of the robot arm 40 having an arc shape is formed under the table 30, and the robot arm 40 is withdrawn from the base such that the fore end of the robot arm 40 can reach for a predetermined area in the table 30. The arc shape leads the withdrawn robot arm 40 to be located above the table 30, thereby facilitating the depositing and withdrawing of the robot arm 40.

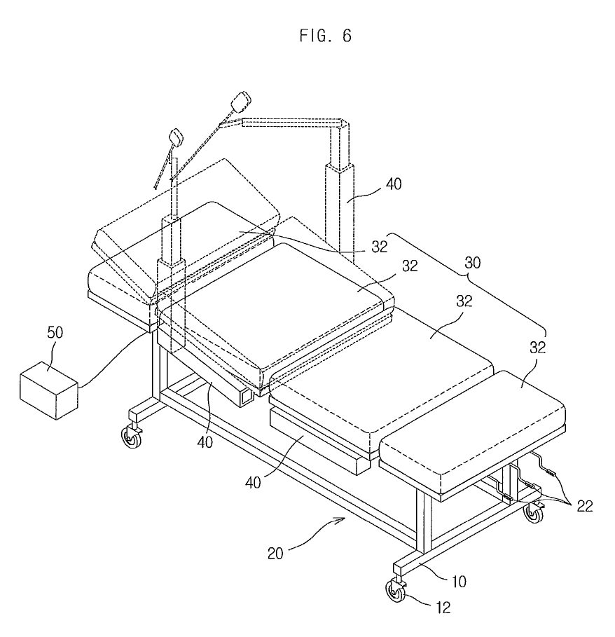

FIG. 6 is a perspective view of a surgical robot integrated with patient bed according to another embodiment of the present invention. In FIG. 6 are illustrated a supporting part 10, a moving means 12, a frame 20, a height adjustment means 22, a table 30, a sub table 32, a robot arm 40, and a main body part 50.

The table 30 of this embodiment may be comprised of a single plate or a plurality of sub tables 32.

The table 30 may be comprised of a plurality of sub tables 32, as shown in FIG. 6, in order to adjust the position of a specific part of the patient.

In this case, a pivot means may be combined with the frame 20 in order to rotate and adjust each sub table 32. For example, when the patient is required to head-up tilted to the body, the sub table 32 supporting the head is elevated and rotated to the body, for which the pivot means for the head supporting sub table 32 is driven.

The pivot means for each sub table 32, like the height adjustment means 22, may operate manually, automatically, or the like.

Additionally, the robot arm 40 may be configured to pivot in synchronization with the pivoting of each sub table 32, whereby the driving coordinate of the robot arm 40 with respect to each sub table can remain unchanged, which allows the robot arm 40 to be manipulated based on the same coordinate regardless of the pivoting of the sub table 32.

In order to realize this, the robot arm 40 may be installed to be connected with the sub tables 32, or an input value corresponding to the pivoting of the sub table 32 may be stored so that the coordinate may be changed according to the inputted value before the robot arm 40 is operated.

With the above configuration, the robot arm 40 may be manipulated without an additional setting to direct the surgical area, even when the table 30 comprises a plurality of sub tables 32 that are separately pivoted.

Although the present invention is described by referring to one of preferable embodiments, it will be appreciated by those skilled in the art that changes may be made without departing from the principles and spirit of the invention, the scope of which is defined in the appended claims and their equivalents.

1. A bed integrated surgical robot comprising:
a supporting part;
a frame supported by the supporting part;
a table supported by the frame and on which a surgical patient is laid; and
a surgical robot arm installed on the frame and manipulated to reach for the surgical patient,
wherein the robot arm is formed of a plurality of joints and deposited on the frame by driving the plurality of joints.

2. The bed integrated surgical robot according to claim 1, wherein a fore end of the robot arm reaches for a predetermined area of the table by driving the plurality of joints.

3. The bed integrated surgical robot according to claim 1, wherein the supporting part comprises a moving means for moving the robot.

4. The bed integrated surgical robot according to claim 1, wherein the frame is combined with a height adjustment means for adjusting a height of the table.

5. The bed integrated surgical robot according to claim 4, wherein a height of the robot arm is adjusted in synchronization with the table.

6. The bed integrated surgical robot according to claim 1, wherein the table is formed of a plurality of sub tables and a pivot means is combined with the frame to separately pivot the plurality of sub tables in correspondence with a posture of the patient.

7. The bed integrated surgical robot according to claim 6, the robot arm is pivoted in synchronization with the sub table, whereby the fore end of the robot arm reaches for a predetermined area of the sub table.

8. The bed integrated surgical robot according to claim 1, the surgical robot further comprising a main body part that is connected with the robot arm and transmits signal for driving the plurality of joints.

9. The bed integrated surgical robot according to claim 8, wherein the main body part transmits signal corresponding to a deposit mode, and when the robot arm receives the signal corresponding to the deposit mode the plurality of joints are driven to deposit the robot arm on the frame.

10. The bed integrated surgical robot according to claim 8, wherein the main body part transmits signal corresponding to an operation mode, and when the robot arm receives the signal corresponding to the operation mode the plurality of joints are driven such that the fore end of the robot arm reaches for a predetermined area of the table.
